(19) United States
(12) Patent Application Publication
(10) Pub. No.: US 2024/0409112 A1
(43) Pub. Date: Dec. 12, 2024

(54) Method for Assisting a Vehicle User During a Manoeuvre of the Vehicle on a Multi-Lane Road Taking Into Account an Individual Reaction Time of the User, Driver Assistance System, and Vehicle

(71) Applicant: Bayerische Motoren Werke Aktiengesellschaft, Muenchen (DE)

(72) Inventors: Sami FAYAD, Muenchen (DE); Stephan NIERMANN, Hebertshausen (DE); Dominik RIETH, Muenchen (DE)

(21) Appl. No.: 18/702,845

(22) PCT Filed: Sep. 30, 2022
(86) PCT No.: PCT/EP2022/077269

§ 371 Date: Apr. 19, 2024

(30) Foreign Application Priority Data
Oct. 29, 2021 (DE) 10 2021 128 328.0

(51) Int. Cl. B60W 50/14; A61B 5/18; G08G 1/16

(52) U.S. Cl. CPC B60W 50/14; A61B 5/18; G08G 1/167; B60W 2540/229

(57) ABSTRACT

A method for assisting a user of a vehicle during the maneuvering of the vehicle on a multi-lane road includes receiving a travel command to maneuver the vehicle from a second lane of the road across a first lane of the road onto an exit of the road, searching for a free gap for the vehicle between road users in the first lane, issuing an indication to the user to actuate an operator control element to initiate a lane change maneuver from the second lane into the free gap in the first lane, wherein the indication is issued at a certain indication time, determining a total reaction time which describes a duration between the issuing of the indication and the initiation of the lane change maneuver, determining an individual reaction factor of the user based on the total reaction time and adapting the indication time for subsequent maneuvering of the vehicle on the basis of the individual reaction factor.

### BACKGROUND AND SUMMARY

The present invention relates to a method for assisting a user of a vehicle during the maneuvering of the vehicle on a multi-lane road. In addition, the present invention relates to a driver assistance system. Finally, the present invention relates to a vehicle having such a driver assistance system.

Driver assistance systems for carrying out at least partially automated lane change maneuvers, or so-called lane change assistance systems, are known from the prior art. In the case of these driver assistance systems having an automated or automatic lane change function, the user or driver typically indicates a lane change by way of a certain operator control action. When such an operator control action is detected, the driver assistance system is used to maneuver the vehicle along a planned trajectory into a neighboring lane or the target lane with automatic lateral guidance and generally also with automatic longitudinal guidance. In the case of such driver assistance systems or lane change assistance systems, the driver's wish to change lane is usually signaled by the actuation of a corresponding operator control element, for example of a turn signal lever, for activating turn signals visible on the outside of the vehicle.

Known driver assistance systems or lane change assistance systems generally use a suitable environment sensor system to monitor the environment of the vehicle before the lane change. In response to the operator control action by the driver, the lane can then be changed if a free gap for the ego vehicle has been detected in the neighboring lane.

Moreover, driver assistance systems which assist the user of the vehicle on a multi-lane road in reaching an exit are known from the prior art. In this case, the driver assistance system can, for example, as a result of a navigation target being input, prepare all necessary lane changes up to reaching the exit. For this purpose, the driver assistance system can initially detect a free gap for the vehicle in the target lane, and subsequently adapt the speed of the vehicle for the subsequent lane change maneuver into the detected gap. The lane change itself can then be carried out by means of a lane change assistance system, wherein the lane change assistance system is initiated automatically or following an operator control input by the user.

When, for example, a free gap for the ego vehicle has been detected in the neighboring lane or the target lane and/or the speed of the vehicle for the subsequent lane change maneuver has been correspondingly adapted, a corresponding indication to initiate the lane change maneuver is issued to the user. As a result of this indication, the user can then actuate a corresponding operator control element, for example, the turn signal lever, and thus initiate the at least partially automated lane change maneuver. According to the prior art, this lane change indication is issued at a previously defined, fixed time.

It is an object of the present invention to present a solution as to how operation of a driver assistance system of the aforementioned type can be adapted to the user. In addition, a vehicle having a corresponding driver assistance system is intended to be provided.

This object is achieved according to the invention by a method, by a driver assistance system and by a vehicle having the features according to the independent claims. Advantageous developments of the present invention are specified in the dependent claims.

A method according to the invention serves to assist a user of a vehicle during the maneuvering of the vehicle on a multi-lane road. The method comprises receiving a travel command to maneuver the vehicle from a second lane of the road across a first lane of the road onto an exit of the road. Moreover, the method comprises searching for a free gap for the vehicle between road users in the first lane. Additionally, the method comprises issuing an indication to the user to actuate an operator control element to initiate a lane change maneuver from the second lane into the free gap in the first lane, wherein the indication is issued at a certain indication time. Furthermore, the method comprises determining a total reaction time which describes a duration between the issuing of the indication and the initiation of the lane change maneuver. Additionally, the method comprises determining an individual reaction factor of the user based on the total reaction time and adapting the indication time for subsequent maneuvering of the vehicle on the basis of the individual reaction factor.

The method intends to assist the user or the driver of the vehicle during the maneuvering of the vehicle on the multi-lane road. The multi-lane road can in principle be a highway, a federal road, an expressway, a freeway-like road or the like. However, the multi-lane road is preferably a freeway. The road can, for example, have two roadways each having at least two lanes which are referred to as first lane and second lane in the present case. Both the ego vehicle and other road users can move in these lanes in a predefined direction of travel. The road can also have further lanes. In this case, the first lane can adjoin the exit or a deceleration lane leading to the exit. The first lane can, for example, be the right lane of a freeway.

The driver assistance system is used to receive the travel command which describes that the vehicle should be maneuvered from the second lane across the first lane onto the exit of the road. In other words, a lane change from the second lane into the first lane should thus initially be carried out and the vehicle should subsequently leave the road via the exit. Furthermore, the vehicle can change from the multi-lane road to another road via the exit. This is the case, for example, for a freeway interchange or the like. In principle, the travel command can be predefined by a route guidance system or a navigation system of the vehicle. Alternatively or in addition, the travel command can be established by a corresponding operator control input by the user.

Moreover, the driver assistance system can be used to prepare the lane change or the lane change maneuver from the second lane into the first lane. For this purpose, corresponding distance sensors or environment sensors of the driver assistance system can initially be used to search for free gaps for the vehicle in the first lane. In addition, the driver assistance system can be used to adapt the speed of the vehicle or the longitudinal speed. In particular, the speed of the ego vehicle can be reduced or adapted to match the speed of the other road users in the first lane. For example, the speed of the vehicle can be adapted in such a way that a gap in the target lane is followed.

Additionally, the indication is issued to the user to actuate the operator control element or the turn signal lever. As a result of the actuation of the operator control element, the lane change maneuver from the second lane to the first lane can then be initiated or commenced. The lane change maneuver can be carried out automatically or automatedly by means of the driver assistance system or a lane change assistance system. Upon initiation of the lane change maneuver, the lane change assistance system can be triggered to carry out the lane change maneuver.

According to the present invention, provision is now made for the total reaction time to be determined. This total reaction time describes the duration between the issuing of the indication to the user and the initiation of the lane change maneuver. The total reaction time is thus that duration which elapses between the request for the user to actuate the operator control element to initiate the lane change maneuver and the initiation of the lane change maneuver by the driver assistance system. Between the issuing of the indication and the initiation of the lane change maneuver, it is necessary for the user to recognize or perceive the indication and subsequently actuate the operator control element.

Based on this total reaction time, the individual reaction factor of the user is now determined. This reaction factor describes in particular the individual or personal reaction behavior of the driver. In the present case, it is taken into account that this individual reaction factor is individual for each user. According to the present invention, provision is made for this individual reaction factor to be learned for the user or also for different users during operation of the vehicle or over the course of time. In particular, a learning system which can determine the individual reaction factor should be provided or used.

In other words, the individual reaction factor can be determined continuously and taken into account during subsequent maneuvering of the vehicle on a multi-lane road. In the present case, the term “subsequent maneuvering” should be understood to mean in particular a driving maneuver which temporally follows the current driving maneuver and in which a lane change maneuver from the second lane to the first lane is prepared and the speed of the ego vehicle is adapted for this purpose. Overall, therefore, the indication time and thus the functionality of the driver assistance system can be continuously adapted to the individual user or driver and the acceptance for the function can thus be improved.

According to the prior art, the indication is issued to the user, for example, as soon as a gap for the vehicle has been found. On the basis of the individual reaction factor, the issuing of the indication can take place earlier or later. In the case of users or drivers with a slower reaction time, the indication can thus be issued earlier than provided as standard. In this way, it can be ensured that the lane change maneuver is carried out in good time. This is highly relevant in particular when reaching the exit or the deceleration lane assigned to the exit. In particular, it can thus be ensured that exits having a short deceleration lane and/or exits without deceleration lanes, such as, for example, so-called tapered exits in the USA, can also be reached safely.

Preferably, the total reaction time comprises a user reaction time, a user action time and a known system time. Here, in order to determine the individual reaction factor, the user reaction time and the user action time are measured, wherein the sum of the user reaction time and of the action reaction time corresponds to a duration between the issuing of the indication and the actuation of the operator control element by the user. During operation of the driver assistance system, the duration between the issuing of the indication by the driver assistance system and the subsequent actuation of the operator control element or of the turn signal lever by the user can be measured. This duration corresponds to the sum of the user reaction time and of the user action time. The user reaction time can also be referred to as reaction time of the user or as mental reaction time. The user reaction time describes the duration or the period of time between the recognition of the indication by the user and the start of the action directed thereby. The user action time describes the duration which the user requires to subsequently actuate the operator control element or the turn signal lever. The system time describes that duration which the driver assistance system requires between the actuation of the operator control element by the user and the initiation of the lane change maneuver. This system time is known or can be ascertained in tests. The individual reaction factor of the user can thus be ascertained precisely on the basis of the user reaction time, the user action time and the known system time.

In a further embodiment, an alertness and/or a tiredness of the user is determined and the individual reaction factor is determined on the basis of the determined alertness and/or tiredness. The alertness and/or the tiredness of the user or of the driver can be ascertained using known methods. For example, the data from a passenger compartment camera and subsequent image processing can be used for this purpose. Alternatively or in addition, use can also be made of biometric sensors or the like in order to be able to ascertain the alertness and/or the tiredness of the driver. According to the present invention, it is taken into account that both the user reaction time and the user action time are each individual for the user but are also dependent on the alertness and/or tiredness of the user.

In order to now be able to accurately determine the individual reaction factor of the user, the alertness and/or the tiredness of the user is ascertained and the determined alertness and/or tiredness of the user is taken into account during the determination of the individual reaction factor based on the user reaction time and the user action time. For example, a fixed scaling factor can be assumed for the tiredness and/or the alertness of the user. For example, a reaction time which is 50% longer can be stipulated at a tiredness of 100%. In this way, the individual reaction factor can be ascertained using little computational effort.

Furthermore, it is advantageous if the alertness and/or the tiredness is determined continuously during operation of the vehicle and the indication time is determined on the basis of the determined alertness and/or tiredness. Provision can thus additionally be made for the tiredness and/or the alertness of the user to be determined continuously during operation of the driver assistance system or of the vehicle. On the basis of the ascertained tiredness and/or alertness, the indication time at which the indication is issued to the user to actuate the operator control element to initiate a lane change maneuver can then be adapted to the tiredness and/or alertness. For example, at the start of the journey or in the case of an alert user, the indication can in principle be issued later than in the case of a tired user or a user who has already been sitting at the steering wheel of the vehicle for a certain time. The issuing of the indication or the indication time can thus also be adapted to the current alertness and/or tiredness of the user.

The alertness and/or tiredness of the user can additionally be determined on the basis of the current traffic situation in the environment of the vehicle.

In particular, the alertness of the user can be ascertained on the basis of the traffic density. In the case of a low traffic density, it is possible to surmise, for example, a lesser alertness than in the case of dense traffic or of a high traffic density.

In a further configuration, a position of at least one hand of the user is determined and the position of the at least one hand is taken into account during the determination of the individual reaction factor based on the user action time. The position of the at least one hand of the user can be ascertained by means of a passenger compartment camera and/or on the basis of steering wheel sensors. In the present case, it is taken into account that the user action time, that is to say that duration which the user actually requires to actuate the operator control element, is dependent on the position of the hand or of the hands of the user. For example, the user action time in the case where the user has their hand on the steering wheel or near to the operator control element may be shorter than in the case where their hand is actuating another operator control element or their hand is far away from the operator control element or turn signal lever. The individual reaction factor of the user can thus be determined or learned precisely based on the user action time.

Furthermore, it is advantageous if the individual reaction factor of the user is additionally determined based on user data which describe the user and/or driving behavior of the user. These user data can be continuously acquired and stored during operation of the vehicle by the user. The user data can be stored individually for the user or driver. For example, these user data can be stored in a key of the vehicle or in a corresponding memory of the vehicle. These user data can describe the user or driver themselves. For example, the user data can describe an age of the user. For example, it can thus be taken into account that the reaction time of the user increases as age increases. In addition, the user data can also describe driving behavior of the user. For example, the user data can describe whether the user is a rather sporty driver or a cautious driver. Additionally, the user data can describe how experienced the user is at operating the vehicle or at using the driver assistance system. By taking into account these user data, the individual reaction factor can be accurately ascertained.

In a further configuration, the indication time is additionally determined on the basis of an indication type which describes whether the indication is issued visually, audibly and/or haptically. The indication type or indication form can thus describe whether the indication to actuate the operator control element for the initiation of the lane change maneuver is issued visually and/or audibly and/or haptically. Here, it is taken into account that the reaction times for visual, audible and haptic stimuli may differ considerably. The individual reaction time and thus the indication time can thus be learned precisely.

An inventive driver assistance system or assistance system for a vehicle is set up to carry out a method according to the invention and the advantageous configurations thereof. The driver assistance system can be set up to receive a travel command to maneuver the vehicle from a second lane of the road across a first lane of the road onto an exit of the road. The driver assistance system can receive this travel command in particular from a navigation system of the vehicle.

Moreover, the driver assistance system can be set up to search for a free gap for the vehicle between road users in the first lane. The driver assistance system can have at least one environment sensor which can be used to detect the other road users in the environment of the vehicle and in particular in the neighboring lane. In addition, the driver assistance system or a corresponding computing device of the driver assistance system can detect free gaps between the other road users based on the data from the at least one environment sensor. This computing device can be formed by at least one electronic control unit of the vehicle. The computing device can fundamentally have at least one processor and one memory.

Additionally, the driver assistance system can be set up to issue an indication to the user to actuate an operator control element to initiate a lane change maneuver from the second lane into the free gap in the first lane, wherein the indication is issued at a certain indication time. In order to issue the indication, the driver assistance system can have an issuing device. In addition, the driver assistance system can be set up to determine a total reaction time which describes a duration between the issuing of the indication and the initiation of the lane change maneuver. The driver assistance system can have a corresponding measuring device which can be used to measure the duration between the issuing of the indication and the actuation of the operator control element or of the turn signal lever by the user. Additionally, the driver assistance system is set up to determine the individual reaction factor of the user based on the total reaction time and to adapt the indication time for subsequent maneuvering of the vehicle on the basis of the individual reaction factor.

Moreover, the driver assistance system can have a passenger compartment sensor system, for example a passenger compartment camera, which can be used to ascertain an alertness and/or tiredness of the driver. This passenger compartment sensor system can additionally be used to determine the position of the hands of the user.

In addition, the driver assistance system can be used to adapt the longitudinal guidance of the vehicle for preparing the lane change maneuver. For this purpose, the computing device can be used to emit corresponding control signals. The computing device or the driver assistance system can additionally be used to calculate the trajectory for the maneuvering of the vehicle or for the automated lane change maneuver. Furthermore, the computing device can be designed to control the steering or a steering system of the vehicle. This control of the steering may make it possible to take over the lateral guidance of the vehicle during the lane change maneuver. Provision can also be made for the longitudinal guidance of the vehicle to be taken over by the driver assistance system during the lane change maneuver.

A vehicle according to the invention comprises a driver assistance system according to the invention. The vehicle can in particular be in the form of a passenger car.

The preferred embodiments presented with respect to the method according to the invention and the advantages thereof correspondingly apply to the driver assistance system according to the invention and the vehicle according to the invention.

Further features of the invention are evident from the claims, the figures and the description of the figures. The features and combinations of features mentioned above in the description, and the features and combinations of features mentioned below in the description of the figures and/or shown in the figures themselves, cannot only be used in the respectively specified combination but also in other combinations or by themselves without leaving the scope of the invention.

The invention will now be explained in more detail on the basis of preferred exemplary embodiments and with reference to the attached drawings, in which:

### BRIEF DESCRIPTION OF THE DRAWINGS

FIG. 1 shows a schematic illustration of a vehicle which has a driver assistance system for maneuvering a vehicle on a multi-lane road; and

FIG. 2 shows the vehicle according to FIG. 1 in a traffic situation, wherein the vehicle is located on a multi-lane road and a driving maneuver from a second lane of the road to an exit of the road is planned.

### DETAILED DESCRIPTION OF THE DRAWINGS

Identical or functionally identical elements are provided with the same reference signs in the figures.

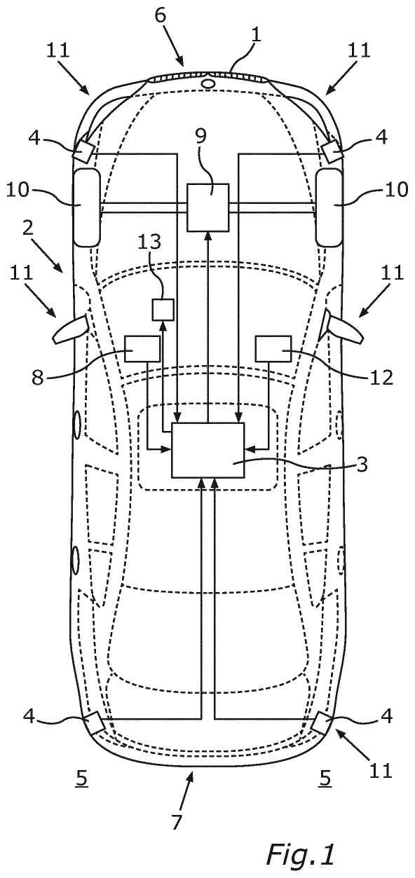

FIG. 1 shows a plan view of a vehicle 1 which is in the form of a passenger car in the present case. The vehicle 1 comprises a driver assistance system 2 which can be used to assist a user of the vehicle 1 during the maneuvering of the vehicle 1 on a multi-lane road 15. The driver assistance system 2 comprises a computing device 3 which can, for example, be formed by at least one electronic control unit of the vehicle 1.

In addition, the driver assistance system 2 comprises at least one environment sensor 4 or distance sensor. In the present example, the driver assistance system 2 comprises four environment sensors 4 of which two environment sensors 4 are arranged in a front region 6 and two environment sensors 4 are arranged in a rear region 7 of the vehicle 1. In the example shown, the environment sensors 4 are arranged in the respective corners of the vehicle 1. The environment sensors 4 can, for example, be in the form of radar sensors. The environment sensors 4 can be used to carry out corresponding measurements in order to be able to detect objects and in particular other road users 14 in an environment 5 of the vehicle 1.

The computing device 3 is further set up to detect an operator control action carried out on an operator control element 8 by the user. The operator control element 8 can in particular be a turn signal lever. As soon as the turn signal lever or the operator control element 8 is actuated by the user, corresponding turn signals 11 of the vehicle 1 can be activated. Moreover, the driver assistance system 2 comprises a passenger compartment sensor system 12 which can, for example, comprise a passenger compartment camera. The passenger compartment sensor system 12 can be used to determine a tiredness and/or alertness of the user. Provision is further made for a position of at least one hand of the user to be ascertained by the passenger compartment sensor system 12. The driver assistance system 2 further comprises an issuing device 13 which can be used to issue an indication to the user of the vehicle 1.

In addition, the computing device 3 is set up to control a steering system 9 of the vehicle 1, which is illustrated only schematically in the present case. Controlling the steering system 9 can make it possible to take over the lateral guidance of the vehicle 1 during a lane change maneuver. In this case, the steerable wheels 10 of the vehicle can be steered by controlling the steering system 9. Preferably, provision is made for the computing device 3 to be able to be additionally used to control a drive motor and/or a brake system of the vehicle 1 in order to take over the longitudinal guidance of the vehicle 1.

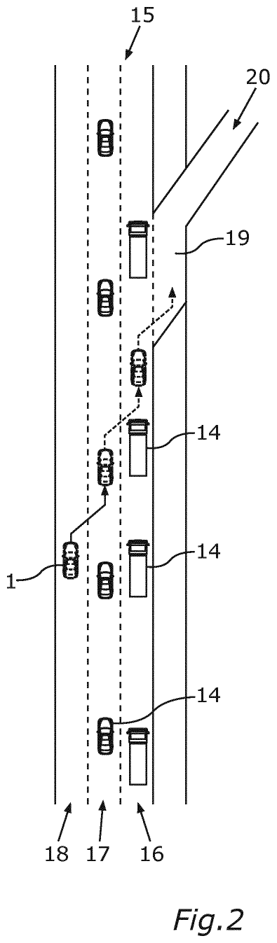

FIG. 2 shows a schematic illustration of a traffic situation in which the vehicle 1 is located on a multi-lane road 15. In the present example, the multi-lane road is in the form of a freeway. The road 15 fundamentally comprises at least two lanes 16, 17. In the present example, the road 15 comprises three lanes 16, 17, 18. Only one roadway of the road 15 is shown in the present case. In this case, the vehicle 1 is currently located in the left lane or a third lane 18. The road 15 additionally comprises a deceleration lane 19. This deceleration lane 19 is assigned to an exit 20.

In the present case, it is planned or is predefined by a travel command that the vehicle 1 will maneuver from the third lane 18 across the second lane 17, the first lane 16 and the deceleration lane 19 onto the exit 20. The driver assistance system 2 is used to assist the user of the vehicle 1 during the respective lane change maneuvers. For this purpose, the driver assistance system 2 is used to search for free gaps between the road users 14 based on the measurements from the environment sensors 4. As soon as a free gap between the road users 14 in the second lane 17 is detected, a speed or longitudinal speed of the vehicle is adapted or reduced. Additionally, at an indication time, the issuing device 13 is used to issue an indication to the driver to actuate the operator control element 8 and thus to initiate the automated lane change from the third lane 18 into the second lane 17. The lane change maneuver from the second lane 17 into the first lane 16 and from the first lane 16 into the deceleration lane 19 are then prepared and subsequently carried out in the same way.

In the present case, provision is made for the issuing of the indication or the indication time to be adapted to the individual reaction time of the user. For this purpose, the total reaction time t_{GR} can be determined according to the following formula:

$${t}_{GR} = {t}_{NR} ( m \ddot{u} d , indiv ) + {t}_{NH} ( m \ddot{u} d , indiv ) + {t}_{sys} .$$

The total reaction time t_{GR} describes the duration between the issuing of the indication by the issuing device 13 and the initiation of the lane change maneuver by the driver assistance system 2. In this case, the total reaction time t_{GR} consists of the user reaction time t_{NR}, the user action time t_{NH} and the system time t_{sys} of the driver assistance system 2. In this case, both the user reaction time t_{NR} and the user action time t_{NH} are, on the one hand, individual for the user and, on the other hand, dependent on the tiredness or alertness of the user. This can be described by the following formula:

$${t}_{GR} = {t}_{NR} \cdot ( {c}_{m \ddot{u} d} + {c}_{indiv} ) + {t}_{NH} \cdot ( {c}_{m \ddot{u} d} + {c}_{indiv} ) + {t}_{sys} .$$

In this case, the factor c_{müd} describes the current tiredness or alertness of the user. This factor can be determined based on the measurements from the passenger compartment sensor system 12. The factor c_{müd} can also be determined based on other factors. For example, in the case of a low traffic density in the environment 5 of the vehicle 1, it is possible to surmise a lesser alertness than in the case of a high traffic density.

Moreover, c_{indiv} describes an individual reaction factor of the user. The user action time t_{NH} is additionally also dependent on the position of the hands of the user. The position of the hands can additionally be detected by the passenger compartment sensor system 12. In the formulae described here, the position of the hands is not taken into account in the present case. Based on the total reaction time t_{GR}, an individual reaction factor c_{indiv} for the user can then be ascertained:

$${c}_{indiv} = \frac{{t}_{GR} - {c}_{m \ddot{u} d} ( {t}_{NR} + {t}_{NH} ) - {t}_{sys}}{{t}_{NR} + {t}_{NH}} .$$

The factors c_{müd} and c_{indiv} can preferably also be determined continuously and be filtered or averaged. The indication time for subsequent driving maneuvers by the vehicle 1 on multi-lane roads 15 can then be adapted based on this individual reaction factor. The individual reaction factor can be determined or learned continuously. The latter can then be assigned to the user and be stored in a user profile or in a vehicle key of the user.

1-9. (canceled)

10. A method for assisting a user of a vehicle during maneuvering of the vehicle on a multi-lane road, the method comprising:
receiving a travel command to maneuver the vehicle from a second lane of the road across a first lane of the road onto an exit of the road;
searching for a free gap for the vehicle between road users in the first lane;
issuing an indication to the user to actuate an operator control element to initiate a lane change maneuver from the second lane into the free gap in the first lane, wherein the indication is issued at an indication time;
determining a total reaction time which describes a duration between the issuing of the indication and the initiation of the lane change maneuver;
determining an individual reaction factor of the user based on the total reaction time; and
adapting the indication time for subsequent maneuvering of the vehicle based on the individual reaction factor.

11. The method according to claim 10, wherein the total reaction time comprises a user reaction time, a user action time and a known system time, wherein in order to determine the individual reaction factor the user reaction time and the user action time are measured and wherein the sum of the user reaction time and of the user action time corresponds to a duration between the issuing of the indication and the actuation of the operator control element by the user.

12. The method according to claim 10, wherein an alertness and/or a tiredness of the user is determined and the individual reaction factor is determined based on the determined alertness and/or tiredness.

13. The method according to claim 11, wherein an alertness and/or a tiredness of the user is determined and the individual reaction factor is determined based on the determined alertness and/or tiredness.

14. The method according to claim 12, wherein the alertness and/or the tiredness is determined continuously during operation of the vehicle and the indication time is determined based on the determined alertness and/or tiredness.

15. The method according to claim 13, wherein the alertness and/or the tiredness is determined continuously during operation of the vehicle and the indication time is determined based on the determined alertness and/or tiredness.

16. The method according to claim 11, wherein a position of at least one hand of the user is determined and the position of the at least one hand is taken into account during the determination of the individual reaction factor based on the user action time.

17. The method according to claim 12, wherein a position of at least one hand of the user is determined and the position of the at least one hand is taken into account during the determination of the individual reaction factor based on the user action time.

18. The method according to claim 10, wherein the individual reaction factor of the user is additionally determined based on user data which describe the user and/or driving behavior of the user.

19. The method according to claim 11, wherein the individual reaction factor of the user is additionally determined based on user data which describe the user and/or driving behavior of the user.

20. The method according to claim 12, wherein the individual reaction factor of the user is additionally determined based on user data which describe the user and/or driving behavior of the user.

21. The method according to claim 13, wherein the individual reaction factor of the user is additionally determined based on user data which describe the user and/or driving behavior of the user.

22. The method according to claim 10, wherein the indication time is additionally determined on the basis of an indication type which describes whether the indication is issued visually, audibly and/or haptically.

23. The method according to claim 11, wherein the indication time is additionally determined on the basis of an indication type which describes whether the indication is issued visually, audibly and/or haptically.

24. The method according to claim 12, wherein the indication time is additionally determined on the basis of an indication type which describes whether the indication is issued visually, audibly and/or haptically.

25. The method according to claim 13, wherein the indication time is additionally determined on the basis of an indication type which describes whether the indication is issued visually, audibly and/or haptically.

26. A driver assistance system for a vehicle, wherein the driver assistance system is configured to carry out a method according to claim 10.

27. A passenger car comprising a driver assistance system according to claim 26.
